(19) United States
(12) Patent Application Publication
(10) Pub. No.: US 2026/0244196 A1
(43) Pub. Date: Aug. 20, 2026

(54) Tracing Back Quality Parameters of Textile Formations

(71) Applicant: Uster Technologies AG, Uster (CH)

(72) Inventors: Heike THOMYS, Egg (CH); Peter Schmid, Zurich (CH); Kurt EGGIMANN, Saland (CH); Sivakumar Narayanan, Uster (CH)

(73) Assignee: Uster Technologies AG, Uster (CH)

(21) Appl. No.: 19/474,937

(22) PCT Filed: Mar. 28, 2024
(86) PCT No.: PCT/CH2024/050016

§ 371 Date: Oct. 13, 2025

(30) Foreign Application Priority Data
Apr. 24, 2023 (CH) CH000424/2023

(51) Int. Cl. G05B 19/418

(52) U.S. Cl. CPC G05B 19/41875; G05B 2219/23101

(57) ABSTRACT

The computer-implemented method serves for tracing back quality parameters of a first textile formation (81). A first textile company (1) transmits a first identifier for the first textile formation (81) and first information comprising a first set of measured values of at least one first quality parameter measured for the first textile formation (81) to a computer system (4) via a global communication network (5). The computer system (4) assigns the first identifier to the first information and stores in a database (44) the first identifier and the first information. A second textile company (2) transmits a request containing the first identifier to the computer system (4). The computer system (4) extracts from the database (44), based on the assignment of the first identifier to the first information, the first information and transmits to the second textile company (2) a response containing the first information.

### FIELD OF THE INVENTION

The present invention lies in the field of textile quality management. It relates to computer-implemented methods and computer systems for tracing back quality parameters of textile formations, according to the independent patent claims. It can be used throughout the whole textile production chain.

### DESCRIPTION OF THE PRIOR ART

Various quality parameters are used in textile quality management for characterizing and monitoring the quality of textile formations such as raw textile fibers, sliver, roving, yarn, fabric, etc. The application report “USTER® LABORATORY SYSTEMS”, Uster Technologies AG, Ed. 4, 2014, lists more than 300 quality parameters describing the various aspects of the quality of fibers and yarns.

Systems for measuring values of textile quality parameters are known. For instance, the brochure “Think Quality™”, Uster Technologies AG, 2022, presents such systems. They are classified into the classes laboratory systems, inline process control systems, fabric inspection systems, etc. Said brochure also contains an overview of part of a textile production chain, namely, a ring-spinning process.

EP-1,138,625 A2 teaches identifying yarn packages by disposing a marking, e.g., a bar code, on a marking surface of a tube serving as a yarn carrier for the yarn package.

CN-110,033,350 A discloses a textile fabric mobile internet transaction platform which comprises an online server, and an application program end, a direct marketing end and a big database which are in communication connection with the online server. The application program end is at least integrated with a supply module and a purchasing module. The basic parameter information of the fabric product is uploaded to an online server, and a unique two-dimensional code is generated. The purchasing module is used for providing classification service, retrieval service and purchasing service for the purchaser. The direct marketing end comprises a fabric offline warehouse for placing and attaching a label. The big database stores the data generated in the operation process of the platform in a classified manner. The online server provides the data interaction and synchronization and pushes different contents according to the trend of the data.

There is a need in the textile industry to know quality parameters of textile formations in the textile production chain without having to measure them. In many cases, such quality parameters can no longer be measured, which is the case in particular for quality parameters of starting or intermediate products that have been further processed in the meantime. For instance, a weaving mill might want to know certain quality parameters of a yarn from which it intends to produce a fabric, or certain quality parameters of raw fibers from which the yarn was spun. In some cases, such quality parameters might no longer be available at the spinning mill. In some cases, it is not possible to assign the available quality parameters to a certain yarn package. In still other cases, the weaving mill does not trust the quality parameters received from the spinning mill in view of potential warranty claims and would prefer to receive the quality parameters from a third, independent instance.

### SUMMARY OF THE INVENTION

It is an object of the present invention to trace back quality parameters of textile formations in the textile production chain.

These and other objects are solved by the computer-implemented methods and computer systems as defined in the independent claims. Advantageous embodiments are specified in the dependent claims.

According to a first aspect, the computer-implemented method according to the invention serves for tracing back quality parameters of a first textile formation. It comprises the following steps: receiving by a computer system via a global communication network from a first textile company a first identifier for the first textile formation and first information comprising a first set of measured values of at least one first quality parameter measured for the first textile formation; assigning by the computer system the first identifier to the first information; storing in a database on the computer system the first identifier and the first information; receiving by the computer system via a global communication network from a second textile company a request containing the first identifier; extracting by the computer system from the database, based on the assignment of the first identifier to the first information, the first information; and transmitting from the computer system via the global communication network to the second textile company a response containing the first information.

According to a second aspect, the computer-implemented method according to the invention serves for tracing back quality parameters of a second textile formation emanating from a first textile formation. It comprises the following steps: receiving by a computer system via a global communication network from a first textile company a first identifier for the first textile formation and first information comprising a first set of measured values of at least one first quality parameter measured for the first textile formation; assigning by the computer system the first identifier to the first information; storing in a database on the computer system the first identifier and the first information; receiving by the computer system via a global communication network from a second textile company a second identifier for the second textile formation; assigning by the computer system the first identifier to the second identifier; storing in the database the second identifier; receiving by the computer system via a global communication network from a third textile company a request containing the second identifier; extracting by the computer system from the database, based on the assignment of the first identifier to the second identifier, the first information; and transmitting from the computer system via the global communication network to the third textile company a response containing the first information.

The computer-implemented method according to the second aspect of the invention can further comprise the following steps: receiving by the computer system via the global communication network from the second textile company second information comprising a second set of measured values of at least one second quality parameter measured for the second textile formation; assigning by the computer system (4) the second identifier to the second information; storing in the database the second information; upon receipt of the request from the third company, extracting by the computer system from the database, based on the assignment of the second identifier to the second information, the second information; and transmitting from the computer system via the global communication network to the third textile company a response containing the second information.

In one embodiment, the first textile formation and/or the second textile formation consists of raw textile fibers. The at least one first quality parameter or the second set of measured values, respectively, can be from the following set: fineness, maturity, micronaire, length, short-fiber content, nep content, strength, foreign-matter content, reflectance, yellowness. The first set of measured values and/or the second set of measured values, respectively, is measured, e.g., by a fiber-testing laboratory instrument. The first information and/or the second information, respectively, can additionally comprise information from the following set: material of the raw textile fibers, plant variety that produced the raw textile fibers, geographical origin of the raw textile fibers, producer of the raw textile fibers, point in time of production of the raw textile fibers, production batch of the raw textile fibers, harvesting method of the raw textile fibers, ginning method of the raw textile fibers, cultivation method of the raw textile fibers.

In one embodiment, the first textile formation and/or the second textile formation is a yarn. The at least one first quality parameter and/or the at least one second quality parameter, respectively, can be from the following set: coefficient of variation of the yarn mass, coefficient of variation of the yarn diameter, hairiness, number of thick places, number of thin places, number and/or length of periodic yarn defects, number of yarn count variations, number of foreign matters, number of splices. The first set of measured values and/or the second set of measured values, respectively, is measured, e.g., by at least one sensor on a yarn-winding machine winding the yarn onto a yarn package. The first information or the second information, respectively, can additionally comprise information from the following set: yarn count, yarn material, fiber processing system, spinning system, envisaged application, amount of yarn on a yarn package, characteristics of the yarn package, producer of the yarn, point in time of production of the yarn, production batch of the yarn.

In one embodiment, the first textile formation and/or the second textile formation is a plane textile formation such as a woven fabric or a knitted fabric. The at least one first quality parameter and/or the at least one second quality parameter, respectively, can be from the following set: yarn density, stich density, color homogeneity, thick and thin yarns, uneven spacing between yarns, broken ends, pattern faults, air permeability, rub fastness, tendency to pilling, color fastness, bending stiffness. The first set of measured values and/or the second set of measured values is measured, e.g., by a fabric-inspection instrument. The first information and/or the second information can additionally comprise information from the following set: weaving pattern, weaving method, knitting pattern, knitting method, envisaged application, amount of plane textile formation on a roll, producer of the plane textile formation, point in time of production of the plane textile formation, production batch of the plane textile formation.

In one embodiment, the first textile formation consists of raw textile fibers and the second textile formation is a yarn manufactured from the raw textile fibers.

In one embodiment, the first textile formation is a yarn, and the second textile formation is a plane textile formation such as a woven fabric or a knitted fabric manufactured from the yarn.

The first and/or second identifier is preferably a globally unique identifier.

The invention also encompasses a computer system comprising means for carrying out the method according to the invention as described above.

The invention further encompasses a computer program having instructions which when executed by a computer system cause the computer system to perform the method according to the invention as described above.

The computer system according to the first aspect of the invention serves for tracing back quality parameters of a first textile formation. It comprises: a receiver for receiving by the computer system via a global communication network from a first textile company a first identifier for the first textile formation and first information comprising a first set of measured values of at least one first quality parameter measured for the first textile formation; a processor for assigning the first identifier to the first information; a memory for storing in a database the first identifier and the first information; a receiver for receiving via a global communication network from a second textile company a request containing the first identifier; a processor for extracting from the database based on the assignment of the first identifier to the first information, the first information; and a transmitter for transmitting from the computer system via the global communication network to the second textile company a response containing the first information.

The computer system according to the second aspect of the invention serves for tracing back quality parameters of a second textile formation emanating from a first textile formation. It further comprises: a receiver for receiving via a global communication network from a first textile company a first identifier for the first textile formation and first information comprising a first set of measured values of at least one first quality parameter measured for the first textile formation; a processor for assigning the first identifier to the first information; a memory for storing in a database the first identifier and the first information; a receiver for receiving via a global communication network from a second textile company a second identifier for the second textile formation; a processor for assigning the first identifier to the second identifier; a memory for storing in the database the second identifier; a receiver for receiving via a global communication network from a third textile company a request containing the second identifier; a processor for extracting from the database, based on the assignment of the first identifier to the second identifier, the first information; and a transmitter for transmitting from the computer system via the global communication network to the third textile company a response containing the first information.

The computer system according to the second aspect of the invention can further comprise: a receiver for receiving via the global communication network from the second textile company second information comprising a second set of measured values of at least one second quality parameter measured for the second textile formation; a processor for assigning the second identifier to the second information; a memory for storing in the database the second information; a processor for extracting from the database, based on the assignment of the second identifier to the second information, the second information; and a transmitter for transmitting via the global communication network to the third textile company a response containing the second information.

The expressions “first”, “second” and “third textile company” denote companies that can but need not differ from each other with respect to their geographic location and their legal dependencies. For instance, the first, second and third textile companies can be the same legal entity with textile plants in different geographic regions, or can be companies independent from each other.

The expressions “first” and “second textile formation” denote textile formations whose appearances and structures can but need not differ from each other. For instance, the first and second textile formations can be a yarn and a fabric, respectively, but alternatively can both be yarns.

The “set of measured values” can consist of any natural number of measured values including one.

A “computer system” as used in this document may consist of several pieces of computer hardware suitably connected for communicating with each other. Such pieces of computer hardware need not necessarily be located at the same site but may rather be distributed over different locations.

The invention makes it possible to trace back quality parameters of textile formations in the textile production chain. Thanks to the invention, a use of a wrong or unsuitable intermediate product, i.e., an intermediate product with unsuitable parameters, for producing a textile product can be avoided. Hence, waste of textile materials is avoided, too. Moreover, the second textile company can do without a reception inspection of the first textile formation and without the measuring apparatus necessary for such an inspection, since the first information received contains the measured values needed. Thus, it saves efforts, costs and space. Likewise, the second company can buy later more of the first textile formation having the same or similar first information for producing an identical textile product, without a reception inspection. Based on the first information, the second company can subdivide a lot purchased and use selected subsets of the lot for certain applications and/or customers.

### BRIEF DESCRIPTION OF THE DRAWINGS

In the following, the invention is explained in detail based on the drawings.

FIGS. 1 and 2 schematically show two embodiments of a computer system according to the invention, together with its environment.

FIG. 3 schematically shows tables of a database implemented in the computer system according to the invention.

### IMPLEMENTATION OF THE INVENTION

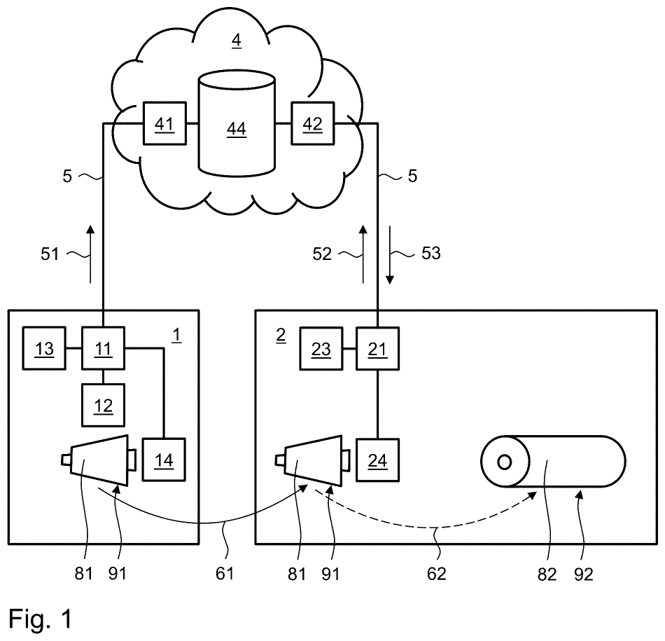

FIG. 1 schematically shows a computer system 4 according to a first embodiment of the invention, together with its environment. The computer system 4 is preferably realized by means of cloud computing, i.e., employs remote shared computer resources, and is therefore symbolized by a cloud in FIG. 1. The computer system 4 is connected via a global communication network 5 such as the world wide web with a first textile company 1 and a second textile company 2. Only one first textile company 1 and one second textile company 2 are drawn in FIG. 1 for the sake of simplicity; however, in practice the numbers of first textile companies 1 and second textile companies 2 connected to the computer system 4 can be significantly higher.

For communicating with the first textile company 1 and the second textile company 2, the computer system 4 is equipped with suitable communication means 41, 42. The communication means 41, 42 include hardware, such as routers, and software, such as application programming interfaces (APIs). They act as a receiver and/or transmitter each.

In the non-limiting example of FIG. 1, the first textile company 1 is a spinning mill producing yarn as a first textile formation 81. The produced yarn 81 is wound on a yarn package 91, e.g., by means of a spinning machine or a winding machine.

The first textile company 1 is equipped with at least one first sensor 12 for measuring values of at least one first quality parameter of the first textile formation 81. The first sensor 12 can be, e.g., part of a yarn-clearing system on a winding machine producing the yarn package. The yarn-clearing system comprises a yarn sensor 12 at each winding position of a winding machine. Alternatively, the at least one first sensor 12 can be part of a spinning machine or a laboratory yarn-testing instrument. The output signals of the first sensor 12 can be evaluated and further processed by an evaluation unit, which is not drawn in FIG. 1 for the sake of simplicity.

Examples of the first quality parameter include coefficient of variation of the yarn mass, coefficient of variation of the yarn diameter, hairiness, number of thick places, number of thin places, number and/or length of periodic yarn defects, number of yarn count variations, number of foreign matters, and number of splices.

Apart from the measured values of the at least one first quality parameter, further information on the first textile formation 81 can be used for characterizing the first textile formation 81. Such further information can be technical and/or non-technical. Taking again yarn as an example for the first textile formation 81, the further information may comprise, e.g., the following:
Yarn count, e.g., Ne 30, etc.;
Yarn material, e.g., cotton, polyester, viscose, modal, wool, etc.;
Fiber-processing system, e.g., carding or combing;
Spinning system, e.g., ring-spun yarn, compact yarn, rotor yarn, air-jet yarn, etc.;
Envisaged application, e.g., knitting or weaving;
Amount of yarn on the yarn package 91, e.g., 10 kg or 500 km;
Characteristics of the yarn package 91, e.g., size (height, diameter), type (conical, cylindrical, etc.), winding type (symmetric, asymmetric, pineapple, etc.), etc.;
Producer of the yarn 81;
Point in time of production of the yarn 81; and/or
Production batch of the yarn 81.

The further information can be inputted, e.g., by means of a first input station 13.

The first textile formation 81 is provided with a first identifier. The first identifier is embodied, e.g., in a bar code, a quick-response (QR) code, a radio-frequency identification (RFID) tag, etc., which can be applied to a tube serving as a yarn carrier for the yarn package 91. The first identifier can be, e.g., a globally unique identifier (GUID). The first identifier is read or inputted at a first identifier-reading station 14.

The first set of measured values of the at least one first quality parameter, together with the further information on the first textile formation 81, if applicable, is referred to as “first information”. The first information and the first identifier are transmitted from the first textile company 1 via the global communication network 5 to the computer system 4, which data transmission is indicated by an arrow 51 in FIG. 1. For this purpose, the at least one first sensor 12, the first input station 13 and the first identifier-reading station 14 can be connected to a first cloud connector 11 connected to the computer system 4 via the global communication network 5. The first cloud connector 11 can comprise a hardware and/or a software. The computer system 4 receives the first identifier and the first information.

The computer system 4 assigns to the received first information the first identifier for the respective first textile formation 81. The first identifier is preferably assigned biuniquely to each received first information. However, in some embodiments of the invention it can be sufficient to assign the same first identifier to sets of measured values for a group of first textile formations 81 presumably having similar properties.

The received first information and the assigned first identifier are stored in a database 44 on the computer system 4.

The first textile formation 81 is shipped from the first textile company 1 to the second textile company 2, which is indicated in FIG. 1 by an arrow 61. The second textile company 2 produces a second textile formation 82, at least partially, from the first textile formation 81, which is indicated in FIG. 1 by an arrow 62. In the non-limiting example of FIG. 1, the second textile company 2 is a weaving mill producing a fabric 82 from the yarn 81 on the yarn package 91, e.g., by means of a weaving machine. The produced fabric 82 is wound on a fabric roll 92. Alternatively, the second textile company 2 can be a knitting mill or any other producer processing yarn 91, or it can be a non-producing entity such as a yarn retailer.

The weaving mill 2 might be interested in obtaining information on the yarn 81 from which it intends to produce or produced the fabric 82, in particular, quality parameters of and further information on the yarn 81. Such first information on the yarn 81 helps the weaving mill 2 to assess whether the yarn 81 is the one they ordered, whether the yarn 81 is suitable for producing the intended fabric 82, etc. Since the information comes from the computer system 4, rather than directly from the spinning mill 1 that produced the yarn 81, it is reliable. For instance, it cannot be subsequently manipulated by the spinning mill 1. In particular, the correctness of the first set of measured values contained in the first information is guaranteed, since it was automatically transmitted to the computer system 4 as measured by the first sensor 12.

For obtaining the first information on the first textile formation 81, the second textile company 2 reads the first identifier on the first textile formation 81 by means of a second identifier-reading station 24. It sends to the computer system 4 via the global communication network 5 a request containing the first identifier. For this purpose, the second identifier-reading station 24 can be connected to a second cloud connector 21 connected to the computer system 4 via the global communication network 5. The second cloud connector 21 can comprise a hardware and/or a software. The sending of the request is indicated in FIG. 1 by an arrow 52.

The computer system 4 receives the request from the second textile company 2. It uses the first identifier contained in the request for extracting from the database 44 the first information, to which the first identifier is assigned.

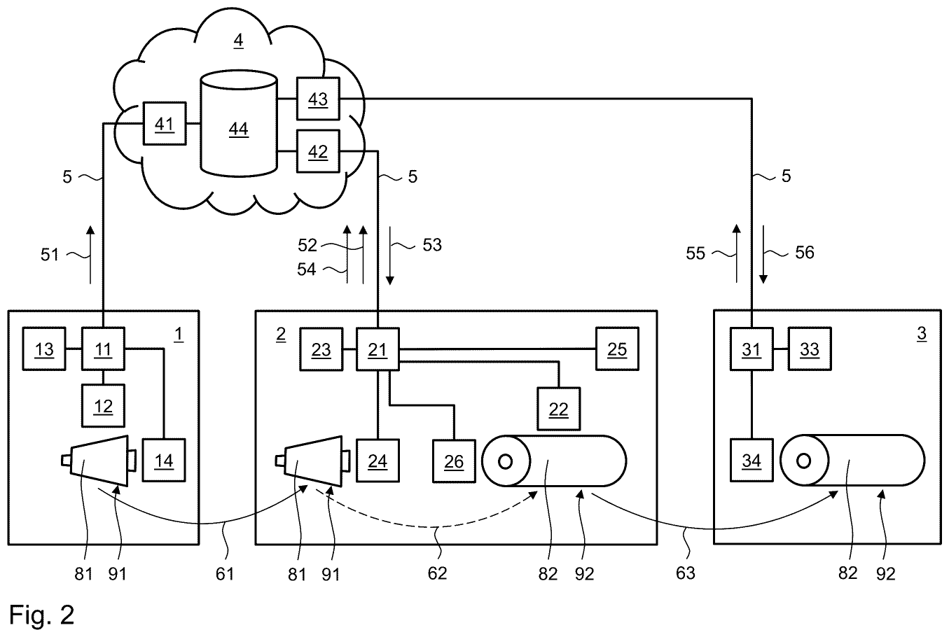

The computer system 4 then transmits to the second textile company 2 via the global communication network 5 a response containing the first information. The transmission of the response is indicated in FIG. 2 by an arrow 53. The second textile company 2 receives the first information and can use it further. The received first information can be outputted in the second textile company 2 by means of a second output station 23 connected to the second cloud connector 21.

FIG. 2 schematically shows a computer system 4 according to a second embodiment of the invention, together with its environment. The elements explained with reference to the first embodiment of FIG. 1 are designated with the same reference signs and need not be discussed again here.

According to the second embodiment, the computer system 4 is additionally connected via a global communication network 5 such as the world wide web with a third textile company 3. Only one third textile company 3 is drawn in FIG. 2 for the sake of simplicity; however, in practice the number of third textile companies 3 connected to the computer system 4 can be significantly higher.

The second textile company 2 is equipped with at least one second sensor 22 for measuring values of at least one second quality parameter of the second textile formation 82. The second sensor 22 can be, e.g., part of an on-loom fault detection system on a weaving machine producing the fabric roll 92. Alternatively, the second sensor 22 can be part of a stand-alone fabric-inspection system. Examples of the second quality parameter include yarn density, stich density, color homogeneity, thick and thin yarns, uneven spacing between yarns, broken ends, pattern faults, air permeability, rub fastness, tendency to pilling, color fastness, and bending stiffness.

Apart from the measured values of the at least one second quality parameter, further information can be used for characterizing the second textile formation 82. Such further information can be technical and/or non-technical. Taking again fabric as an example for the second textile formation 82, the further information may comprise, e.g., the following:
Weaving pattern, e.g., plain weave;
Weaving method, e.g., air-jet weaving;
Knitting pattern, e.g., tricot;
Knitting method, e.g., circular knitting;
Envisaged application, e.g., shirt;
Amount of fabric 82 on the fabric roll 92, e.g., 20 kg or 40 m²;
Producer of the fabric 82;
Point in time of production of the fabric 82; and/or
Production batch of the fabric 82.

The further information can be inputted, e.g., by means of a second input station 25.

The second textile formation 82 is provided with a second identifier embodied, e.g., in a bar code, a quick-response (QR) code, a radio-frequency identification (RFID) tag, etc., which can be applied on the fabric itself or on a tube serving as a fabric carrier for the fabric roll 92. The second identifier can be, e.g., a globally unique identifier (GUID). The second identifier is read or inputted at a third identifier-reading station 26.

The second set of measured values of the at least one second quality parameter, together with the further information on the second textile formation 82, if applicable, is referred to as “second information”. The second information and the second identifier are transmitted from the second textile company 2 via the global communication network 5 to the computer system 4, which data transmission is indicated by an arrow 54 in FIG. 2. For this purpose, the at least one second sensor 22, the second input station 25 and the second identifier reading station 26 can be connected to the second cloud connector 21 connected to the computer system 4 via the global communication network 5. Alternatively, said elements 22, 25, 26 could each be connected to an assigned cloud connector, depending on the cabling and network topology. The computer system 4 receives the second information and the second identifier.

The computer system 4 assigns to the received second information the second identifier for the respective second textile formation 82. The second identifier is preferably assigned biuniquely to each received second information. However, in some embodiments of the invention it can be sufficient to assign the same second identifier to sets of measured values for a group of second textile formations 82 presumably having similar properties.

The received second information and the assigned second identifier are stored in the database 44 on the computer system 4.

A third textile company 3 might be interested in obtaining information on the second textile formation 82 and/or on the first textile formation 81 from which the second textile formation 82 was produced, in particular, quality parameters of and further information on the second textile formation 82 and/or the first textile formation 81. The third textile company 3 can be, e.g., a garment manufacturer manufacturing garments from the fabric 82, or a fabric retailer. Such information on the fabric 82 helps the garment manufacturer to assess whether the fabric 82 is the one they ordered, whether the fabric 82 is suitable for producing the intended garments, etc. Since the information comes from the computer system 4, rather than directly from the weaving mill 2 that produced the fabric 82 or from the spinning mill 1 that produced the yarn 81, it is reliable. For instance, it cannot be subsequently manipulated by the weaving mill 2 or the spinning mill 1. In particular, the correctness of the first set of measured values contained in the first information and of the second set of measured values contained in the second information is guaranteed, since the first and second sets of information were automatically transmitted to the computer system 4 as measured by the first sensor 12 and the second sensor 22, respectively.

For obtaining the information on the second textile formation 82 and/or on the first textile formation 81, the third textile company 3 reads the second identifier on the second textile formation 82 by means of a fourth identifier-reading station 34. It sends to the computer system 4 via the global communication network 5 a request containing the second identifier. For this purpose, the fourth identifier reading station 34 can be connected to a third cloud connector 31 connected to the computer system 4 via the global communication network 5, and the computer system 4 can contain third communication means 43. The sending of the request is indicated in FIG. 2 by an arrow 55.

The computer system 4 receives the request from the third textile company 3. It uses the second identifier contained in the request for extracting from the database 44 the first information and/or the second information, to which the first identifier and/or second identifier, respectively, is assigned.

The computer system 4 then transmits via the global communication network 5 to the third textile company 3 a response containing the first information and/or the second information. The transmission of the response is indicated in FIG. 2 by an arrow 56. The third textile company 3 receives the first information and/or the second information and can use it further. The received first information and/or second information can be outputted in the third textile company 3 by means of a third output station 33 connected to the third cloud connector 31.

In an analogous way, the method according to the invention can be expanded to any discretionary number of textile formations emanating one from the other, e.g., to the three textile formations raw fibers, yarn and fabric.

It should be pointed out that the embodiments discussed with reference to FIGS. 1 and 2 are in no way limiting for the present invention. The first textile formation 81 and the second textile formation 82 can be any textile formation such as raw textile fibers, sliver, roving, yarn, woven or knitted fabric, etc. They can but need not be different textile formations.

In an embodiment in which the first textile formation 81 or the second textile formation 82 consists of raw textile fibers, the first or second quality parameter, respectively, can be at least one parameter from the following set: fineness, maturity, micronaire, length, short-fiber content, nep content, strength, foreign-matter content, reflectance, yellowness. Further information on the raw textile fibers may comprise, e.g., the following:
Material of the raw textile fibers, e.g., cotton, polyester, viscose, modal, wool, etc.;
Plant variety that produced the raw textile fibers, e.g., PHY 881 R cotton, etc.
Geographical origin of the raw textile fibers;
Producer of the raw textile fibers;
Point in time of production of the raw textile fibers, i.e., in case of natural grown fibers, the time of harvest, and in case of animal hair, the time of cut;
Production batch of the raw textile fibers;
Harvesting method of the raw textile fibers, e.g., manual or machine picking;
Ginning method of the raw textile fibers, e.g., roller gin or saw gin;
Cultivation method of the raw textile fibers, e.g., organic-or conventional.

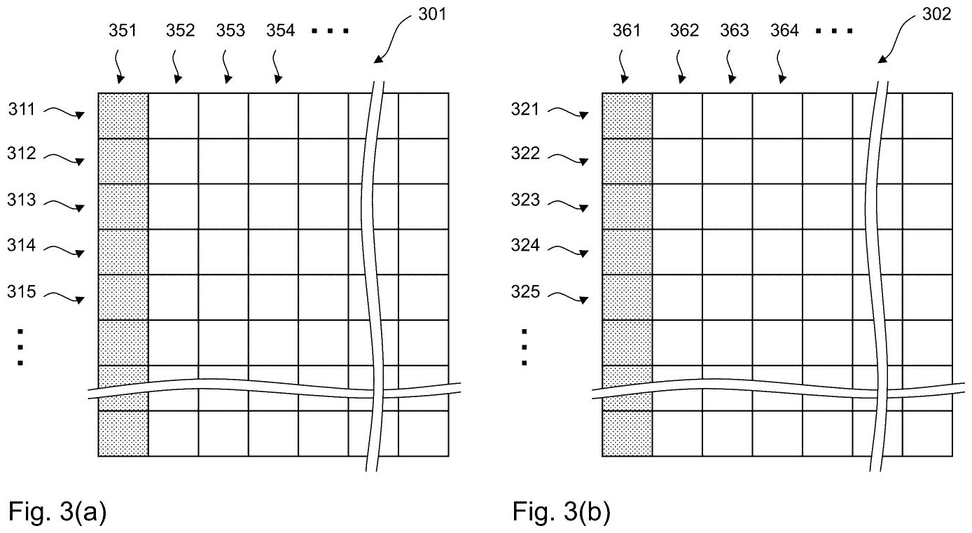

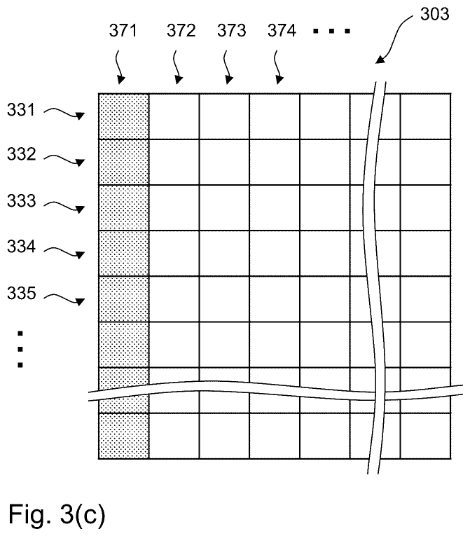

FIG. 3 schematically shows tables 301, 302, 303 of the database 44 implemented in the computer system 4 according to the invention. Each row 311, 312, . . . ; 321, 32, . . . , 331, 332, . . . of the tables 301, 302, 303 contains a tuple of data relating to a certain textile formation.

The first column 351 of the table 301 of FIG. 3(a) contains the first identifiers uniquely identifying the respective first information. The second and subsequent columns 352, 353, . . . contain pieces of the first information for each first textile formation 81. The first information includes the first set of measured values and the further information, if any.

The table 302 of FIG. 3(b) is built up analogously to the table 301 of FIG. 3(a), but for the second textile formations 82 as discussed for the embodiment of FIG. 2. Hence, the first column 361 contains the second identifiers and the second and subsequent columns 362, 363, . . . contain pieces of the second information for each second textile formation 82.

The table 303 of FIG. 3(c) also relates to the embodiment of FIG. 2. Its first column 371 contains the second identifiers, whereas its second and subsequent columns 372, 373, . . . contain the first identifiers related to all those first textile formations 81 from which the second textile formation 82 was manufactured. Referring to the embodiment of FIG. 2, one fabric roll 92 was manufactured from a plurality of yarn packages 91. A row 331, 332, . . . of the table 303 thus assigns all yarn packages 91 involved to the corresponding fabric roll 92. By means of a combination of tables 301 and 303, the first information of all first textile formations 81 involved can be retrieved from the database 44 for each second textile formation 82.

In the embodiment of FIG. 3(a)-(c), it is assumed that the first identifier is assigned biuniquely to each received first information. Thus, the first identifiers in the first column 351 of the table 301 serve as a natural primary key for the table 301. Likewise, the second identifiers in the first columns 361, 371 of the tables 302, 303, respectively, serve as a natural primary key for each of the tables 302, 303. Alternatively, other keys including surrogate keys can be used in the database 44.

The relational database discussed with reference to FIG. 3 is merely an example and shall not limit the generality of the invention. Any other data storage and retrieval mechanism such as a NoSQL database can be used in the present invention.

It is understood that the present invention is not limited to the embodiments discussed above. With knowledge of the invention, the person skilled in the art will be able to derive further variants which are also part of the subject matter of the present invention.

### LIST OF REFERENCE NUMERALS

1 First textile company
11 First cloud connector
12 First sensor
13 First input station
14 First identifier-reading station
2 Second textile company
21 Second cloud connector
22 Second sensor
23 Second output station
24 Second identifier-reading station
25 Second input station
26 Third identifier-reading station
3 Third textile company
31 Third cloud connector
33 Third output station
34 Fourth identifier-reading station
4 Computer system
41, 42, 43 Communication means
44 Database
5 Global communication network
51-56 Data transmission
61 Shipping of first textile formation
62 Production of second textile formation from first textile formation
63 Shipping of second textile formation
81 First textile formation, e.g., yarn
82 Second textile formation, e.g., fabric
91 Yarn package
92 Fabric roll
301-303 Tables of the database 44
311, 312, . . . Rows of the first table 301
321, 322, . . . Rows of the second table 302
331, 332, . . . Rows of the third table 303
351, 352, . . . Columns of the first table 301
361, 362, . . . Columns of the second table 302
371, 372, . . . Columns of the third table 303

1. A computer-implemented method for tracing back quality parameters of a first textile formation (81), comprising the steps of:
receiving by a computer system (4) via a global communication network (5) from a first textile company (1) a first identifier for the first textile formation (81) and first information comprising a first set of measured values of at least one first quality parameter measured for the first textile formation (81);
assigning by the computer system (4) the first identifier to the first information;
storing in a database on the computer system (4) the first identifier and the first information;
receiving by the computer system (4) via a global communication network (5) from a second textile company (2) a request containing the first identifier;
extracting by the computer system (4) from the database (44), based on the assignment of the first identifier to the first information, the first information; and
transmitting from the computer system (4) via the global communication network (5) to the second textile company (2) a response containing the first information.

2. A computer-implemented method for tracing back quality parameters of a second textile formation (82) emanating from a first textile formation (81), comprising the steps of:
receiving by a computer system (4) via a global communication network (5) from a first textile company (1) a first identifier for the first textile formation (81) and first information comprising a first set of measured values of at least one first quality parameter measured for the first textile formation (81);
assigning by the computer system (4) the first identifier to the first information;
storing in a database on the computer system (4) the first identifier and the first information;
receiving by the computer system (4) via a global communication network (5) from a second textile company (2) a second identifier for the second textile formation (82);
assigning by the computer system (4) the first identifier to the second identifier;
storing in the database (44) the second identifier;
receiving by the computer system (4) via a global communication network (5) from a third textile company (3) a request containing the second identifier;
extracting by the computer system (4) from the database (44), based on the assignment of the first identifier to the second identifier, the first information; and
transmitting from the computer system (4) via the global communication network (5) to the third textile company (3) a response containing the first information.

3. The computer-implemented method according to claim 2, further comprising the steps of:
receiving by the computer system (4) via the global communication network (5) from the second textile company (2) second information comprising a second set of measured values of at least one second quality parameter measured for the second textile formation (82);
assigning by the computer system (4) the second identifier to the second information;
storing in the database (44) the second information;
upon receipt of the request from the third company (3), extracting by the computer system (4) from the database based on the assignment of the second identifier to the second information, the second information; and
transmitting from the computer system (4) via the global communication network (5) to the third textile company (3) a response containing the second information.

4. The computer-implemented method according to claim 1, wherein the first textile formation (81) and/or the second textile formation consists of raw textile fibers.

5. The computer-implemented method according to claim 4, wherein the at least one first quality parameter and/or the second set of measured values, respectively, is from the following set: fineness, maturity, micronaire, length, short-fiber content, nep content, strength, foreign-matter content, reflectance, yellowness.

6. The computer-implemented method according to claim 4, wherein the first set of measured values and/or the second set of measured values, respectively, is measured by a fiber-testing laboratory instrument.

7. The computer-implemented method according to claim 4, wherein the first information and/or the second information, respectively, additionally comprises information from the following set: material of the raw textile fibers, plant variety that produced the raw textile fibers, geographical origin of the raw textile fibers, producer of the raw textile fibers, point in time of production of the raw textile fibers, production batch of the raw textile fibers, harvesting method of the raw textile fibers, ginning method of the raw textile fibers, cultivation method of the raw textile fibers.

8. The computer-implemented method according to claim 4, wherein the first textile formation (81) and/or the second textile formation is a yarn.

9. The computer-implemented method according to claim 8, wherein the at least one first quality parameter and/or the at least one second quality parameter, respectively, is from the following set: coefficient of variation of the yarn mass, coefficient of variation of the yarn diameter, hairiness, number of thick places, number of thin places, number and/or length of periodic yarn defects, number of yarn count variations, number of foreign matters, number of splices.

10. The computer-implemented method according to claim 8, wherein the first set of measured values and/or the second set of measured values, respectively, is measured by at least one sensor on a yarn-winding machine winding the yarn onto a yarn package (91).

11. The computer-implemented method according to claim 8, wherein the first information and/or the second information, respectively, additionally comprises information from the following set: yarn count, yarn material, fiber processing system, spinning system, envisaged application, amount of yarn on a yarn package, characteristics of the yarn package, producer of the yarn, point in time of production of the yarn, production batch of the yarn.

12. The computer-implemented method according to claim 8, wherein the first textile formation and/or the second textile formation (82) is a plane textile formation such as a woven fabric or a knitted fabric.

13. The computer-implemented method according to claim 12, wherein at least one first quality parameter and/or the at least one second quality parameter, respectively, is from the following set: yarn density, stich density, color homogeneity, thick and thin yarns, uneven spacing between yarns, broken ends, pattern faults, air permeability, rub fastness, tendency to pilling, color fastness, bending stiffness.

14. The computer-implemented method according to claim 12, wherein the first set of measured values and/or the second set of measured values, respectively, is measured by a fabric-inspection instrument.

15. The computer-implemented method according to claim 12, wherein the first information and/or the second information, respectively, additionally comprises information from the following set: weaving pattern, weaving method, knitting pattern, knitting method, envisaged application, amount of plane textile formation on a roll, producer of the plane textile formation, point in time of production of the plane textile formation, production batch of the plane textile formation.

16. The computer-implemented method according to claim 4, wherein the first textile formation consists of raw textile fibers and the second textile formation is a yarn manufactured from the raw textile fibers.

17. The computer-implemented method according to claim 8, wherein the first textile formation (81) is a yarn and the second textile formation (82) is a plane textile formation such as a woven fabric or a knitted fabric manufactured from the yarn.

18. The computer-implemented method according to claim 1, wherein the first and/or second identifier is a globally unique identifier.

19. A computer system (4) comprising means for carrying out the method according to claim 1.

20. A computer program having instructions which when executed by a computer system (4) cause the computer system (4) to perform the method according to claim 1.

21. A computer system (4) for tracing back quality parameters of a first textile formation (81), comprising:
a receiver (41) for receiving by the computer system (4) via a global communication network (5) from a first textile company (1) a first identifier for the first textile formation (81) and first information comprising a first set of measured values of at least one first quality parameter measured for the first textile formation (81);
a processor for assigning the first identifier to the first information;
a memory for storing in a database (44) the first identifier and the first information;
a receiver for receiving via a global communication network (5) from a second textile company (2) a request containing the first identifier;
a processor for extracting from the database (44) based on the assignment of the first identifier to the first information, the first information; and
a transmitter (42) for transmitting from the computer system (1) via the global communication network (5) to the second textile company (2) a response containing the first information.

22. A computer system (4) for tracing back quality parameters of a second textile formation (82) emanating from a first textile formation (81), further comprising:
a receiver (41) for receiving via a global communication network (5) from a first textile company (1) a first identifier for the first textile formation (81) and first information comprising a first set of measured values of at least one first quality parameter measured for the first textile formation (81);
a processor for assigning the first identifier to the first information;
a memory for storing in a database (44) the first identifier and the first information;
a receiver (42) for receiving via a global communication network (5) from a second textile company (2) a second identifier for the second textile formation (82);
a processor for assigning the first identifier to the second identifier;
a memory for storing in the database (44) the second identifier;
a receiver (43) for receiving via a global communication network (5) from a third textile company (3) a request containing the second identifier;
a processor for extracting from the database (44), based on the assignment of the first identifier to the second identifier, the first information; and
a transmitter (43) for transmitting from the server computer (1) system via the global communication network (5) to the third textile company (3) a response containing the first information.

23. The computer system (4) according to claim 22, further comprising:
a receiver (42) for receiving via the global communication network (5) from the second textile company (2) second information comprising a second set of measured values of at least one second quality parameter measured for the second textile formation (82);
a processor for assigning the second identifier to the second information;
a memory for storing in the database (44) the second information;
a processor for extracting from the database based on the assignment of the second identifier to the second information, the second information; and
a transmitter (43) for transmitting via the global communication network (5) to the third textile company (3) a response containing the second information.
